(19) United States
(12) Patent Application Publication
(10) Pub. No.: US 2009/0240180 A1
(43) Pub. Date: Sep. 24, 2009

(54) Mechanical Trauma Protection device

(76) Inventor: Edward Kaine, Stratford, CT (US)

Correspondence Address: Edward Cedric Kaine, 166 Holmes St, stratford, CT, 06615, US

(21) Appl. No.: 12/053,542

(22) Filed: Mar. 21, 2008

(51) Int. Cl. A61F 5/00; A61F 13/00

(52) U.S. Cl. 602/3; 128/888

(57) ABSTRACT

The mechanical trauma protection device is an arching bar which covers over an open splint to protect and area from mechanical trauma. It can be fabricated from plastic, metal or other material. The arching bar attaches to a splint, brace or other device to prevent contact. It can be made integral to a splint such as a protective posture hand splint by forming the material into an arch which extends from the index finger ray to the wrist. This allows the use of the bar as a handle (preventing patient handling/contact injuries) and to serve as a frame for further barriers.

### FIELD OF THE INVENTION

The invention relates generally to an injury splint protector. More specifically, the invention relates to an arching bar which covers over an open splint to protect and area from mechanical trauma.

### BACKGROUND OF THE INVENTION

The device is an arching bar which covers over an open splint to protect and area from mechanical trauma. It can be fabricated from plastic, metal or other material. The arching bar attaches to a splint, brace or other device to prevent contact. It can be made integral to a splint such as a protective posture hand splint by forming the material into an arch which extends from the index finger ray to the wrist. This allows the use of the bar as a handle (preventing patient handling/contact injuries) and to serve as a frame for further barriers.

The bar can also be retrofit to a splint by riveting or removable screws (for re-use and cleaning). An item of this type would be cleanable and/or autoclavable depending on material used. This bar is also appropriate for other applications which require an additional barrier to prevent contact, including over surgical sites, external fixation, shunts, fistula or other areas when contact trauma may cause significant injury.

The bar can also be incorporated into clothing to prevent contact over an area not amenable to splinting (e.g. dialysis shunts which are highly vulnerable to grasping or other trauma.) Similar to but lighter than padding, the device is then extended to cover an area which would otherwise be at risk without touching the surface at risk. This can be as integral to the clothing or as a breakaway device in the event of trauma.

Originally conceived and proven efficacious in hand splinting to minimize contact and mechanical trauma.

Risks associated with having direct contact of a hard splint to skin have often necessitated open bandaging over wounds, post-surgical surfaces and other applications. In several situations, particularly post surgical grafting and stitching, mechanical trauma to the wound within critical times can result in failure of a surgery. This was originally conceived to protect fragile hand grafts from mechanical trauma against the bed rail. I published a paper on the subject in January in the Journal of Burn Care and Research proving it's efficacy in preventing contact of the grafted hand to the bed rail.

On further review the indications and benefits for other post surgical and protective applications became clear. These include areas to which surface contact would be very undesirable as in dialysis shunting, areas of wounds, closure by secondary intention and skin closure with stapling or sutures. This also prevents possible positioning problems for unconscious or semi-conscious patients such as the accidental slipping of a limb into the space between the bed-rail and the mattress or other confined spaces.

There was no other device available that was similar to this in the literature or available for purchase at the time of publication. This is a necessary preventative means of protecting a site and has potential to aid in several areas to prevent contact to an area which cannot be “armored” due to contact concerns.

This is a simple arch which rests above an area which needs to be protected from contact. The arch can be integral to a pre-fabricated splint or as a reusable device which can be cleaned and attached to subsequent splints. The device can also be incorporated into other holding devices to prevent contact to other areas when the protective device must be away from the body.

“Roll-Bar” for Protective Posture Splint Limits Potential Trauma to Dorsal Hand Grafts Edward C. Kaine, BScOT,* Phillip Fidler, MD,† John Schulz, MD,† Shaher Kahn, MD,† Roselle Crombie, MD,† Sally Dalton, RN,† Andrea Warren, MScOT,* Eric LaBonte, MScPT,* John Palmer, PA-C,† Paul Possenti, PA-C,† Jacqueline Laird, RN,† Nabil Atweh, MD†

The expectation of excellent functional and cosmetic outcomes adds to the challenges of managing the burned hand. The initial fragility of the grafted surface warrants extra measures of protection. A “roll-bar” attached to a splint over a grafted area can serve as protection against mechanical trauma. Two “intrinsic plus” protective posture splints were fabricated;

one had a roll-bar extending from the D2 ray to the distal forearm. Three simulated patients wearing each of the splints attempted to contact the bed rail from supine. Pictures, transferred ink, observation, and subjective comments were used to establish percentage of the surface at risk because of bed rail contact and its ease. Without the roll-bar 100% of the dorsal surface of the hand was accessible to contact with the bed rail. With the roll-bar all subjects were prevented from contact to the dorsum of the hand and contact to the dorsal fingers was less than 40% in all subjects, decrease of risk at the wrist was also significant.

The roll-bar can prevent mechanical trauma to grafts on the fingers and dorsum of the hand because of contact with the bed rail. The ease of the application and the potential benefits to patient outcome make it an appropriate addition to the protective posture splint when seeking to minimize area of the surface at risk. (J Burn Care Res 2008; 29:204-207) The expectation of excellent functional and cosmetic outcomes adds to the challenges of managing the burned hand. The initial fragility of the grafted surface warrants extra measures of protection. A “Rollbar” attached to a splint over a grafted area can serve as protection against mechanical trauma. We hypothesize that the roll-bar can reduce area of the dorsal hand available to physical contact and prevent contact with the bed rail (FIG. 1). We sought to quantify the protective effect of the roll-bar on the area of surface at risk.

In our review of the literature, no previous reports of a similar protective device have been published.

### Methods

We fabricated two “intrinsic plus” protective posture splints one with a roll-bar extending from the D2 ray to the distal forearm crossing over the dorsum of the hand (FIG. 2). In both splints a low temperature thermoplastic was used. Standard splinting tools were used including heavy-duty scissors and a splint pan for heating water. The thermoplastic used was noted to be ⅛ in thick to maximize rigidity and increase the tolerance of stretch for the roll-bar (see Appendix). The splints were wrapped on with a 3-in elasticized gauze roll and a gauze roll with a circumferential wrap and diagonally anchored. Three simulated patients wearing the splints attempted to contact the bed rail from a supine position. Blue ink on the bed rail transferred to the bandage when contact was made. In areas where ink transfer was not adequate for confirmation, the surface at risk was verified by repeating the movement with close observation. Pictures and subjective comments were used to determine the ease of bed rail contact.

Pictures of the bandages were compared with the determined percentage of the surface at risk.

### Lateral, From the *Department of Acute Rehabilitation; †Department of Trauma/Burns, Bridgeport Hospital, Bridgeport, Conn.

Address correspondence to Edward C. Kaine, BSCOT, Acute Rehabilitation, Bridgeport Hospital, 267 Grant Street, Bridgeport, Conn. 06610.

### Copyright © 2008 by the American Burn Association.

### 1559-047X/2008

DOI: 10.1097/BCR.0b013e318160d04f
medial, and volar surfaces were removed from the analysis. Fingers were defined as starting at the metacarpophalangeal joint. The dorsum included the dorsal skin from the radial carpal joint to the metacarpophalangeals.

The wrist was defined as the 5 cm proximal to the radial carpal joint. Statistical analysis for significance was performed with a one-way analysis of variance from summary data. The confidence interval was set at 95% and the criteria for significance was P_{—}0.05.

### Results

The roll-bar prevented contact to the dorsum of the hand (FIG. 2). Two of the subjects were able to make contact with the bedrail on several fingers. The wrist also had a reduction of the area at risk. In the cases where the simulated patients were able to make contact with the dorsum of the fingers they rated the movement as difficult and were observed to be in extremes of the available ranges of motion. In the standard open splint each subject made contact with the entire dorsum of the hand and arm to the bed rail.

The findings were statistically significant (Table 1).

Both splints allowed contact to the dorsal the nareminence to the bed rail in either overpronation or oversupination. The forearm and thumb were not protected by this design.

Splinting in burn rehabilitation is a common clinical practice. Few studies have been conducted to validate this practice.1 Immobilization in protective position can maintain the joint ligaments and supporting structures under maximal stretch in an effort to minimize shortening in the presence of edema and inflammation.

2 No prior studies have been published on the use of the roll-bar. It has been used successfully in our center during the past 5 years. Shear stress is one potential mechanism of graft loss after skin grafting.3 A single forceful contact with a bed rail may cause movement of the graft, interrupt the nutrient supply, and lead to failure of

### SUMMARY OF THE INVENTION

The roll-bar can limit the area of contact to a dorsal hand graft. The roll-bar significantly reduces area of contact to objects such as the bed rail. This will reduce potential exposure to shear and other mechanical trauma. The roll-bar can be easily applied to the traditional protective posture splint. The application can be customized to fit the needs of patients for more specific protection of certain areas. Also, the roll-bar can be used as a platform for further protection, as in the application of the ridgeline and rafter wrap. The ease of the application and the potential benefits to graft security seem to justify routine utilization and is our practice with grafting of the hand.

### BRIEF DESCRIPTION OF THE DRAWINGS

The foregoing summary, as well as the following detailed description of the preferred embodiments, is better understood when read in conjunction with the appended drawings. For the purpose of illustrating the invention, there are shown in the drawings embodiments that are presently preferred, it being understood, however, that the invention is not limited to the specific apparatus, system, and instrumentalities disclosed. In the drawings:

FIG. 1:

FIG. 2:

### BRIEF DESCRIPTION OF ILLUSTRATIONS

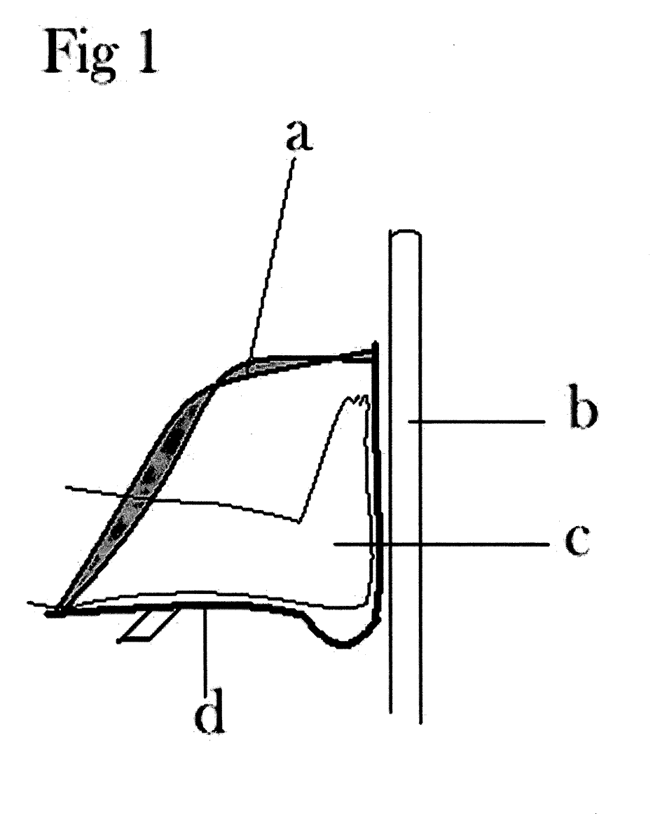

FIG. 1. FRONT VIEW OF THE DEVICE INCORPORATED INTO ANKLE/FOOT SPLINT

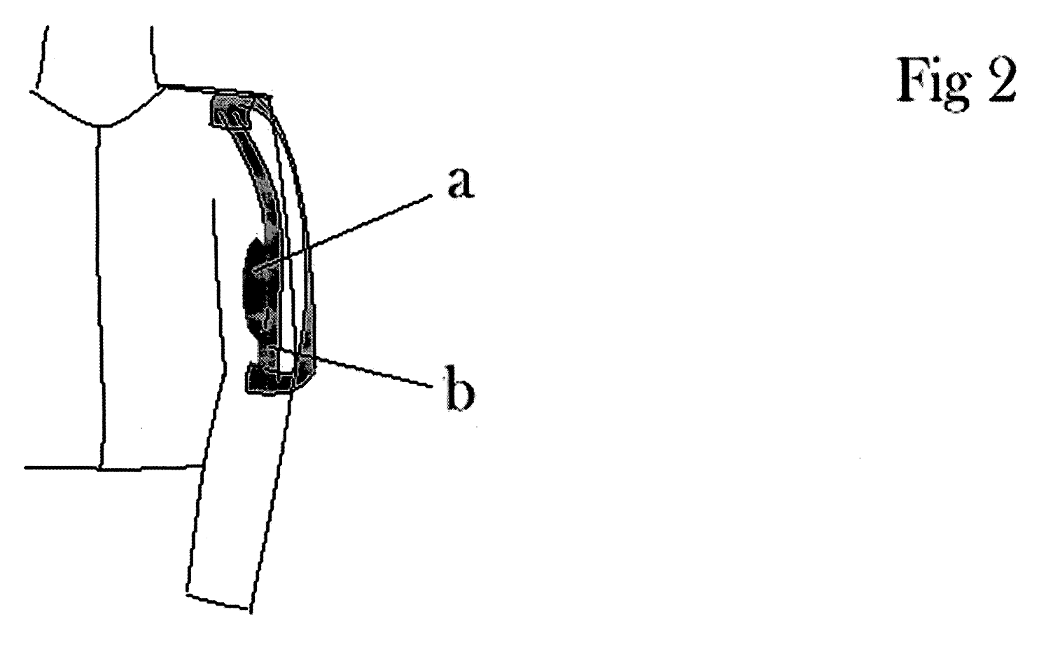

FIG. 2. FRONT VIEW OF THE DEVICE ATTACHED TO ARTICLE OF APPAREL

It is to be understood that the foregoing illustrative embodiments have been provided merely for the purpose of explanation and are in no way to be construed as limiting of the invention. Words which have been used herein are words of description and illustration, rather than words of limitation. Further, although the invention has been described herein with reference to particular structure, materials and/or embodiments, the invention is not intended to be limited to the particulars disclosed herein. Rather, the invention extends to all functionally equivalent structures, methods and uses, such as are within the scope of the appended claims. Having the benefit of the teachings of this specification, others may affect numerous modifications thereto and changes may be made without departing from the scope and spirit of the invention in its aspects.

### DETAILED DESCRIPTION OF ILLUSTRATIVE EMBODIMENTS

FIG. 1) Roll Bar incorporated into Ankle/Foot splint: This represents A single arch or crossed bar extending from the distal sole of the splint back over the ankle to the area above the to the mid calve area. The device will prevent inadvertent or other mechanical trauma from contacting the shielded area.

FIG. 1 Details: a) Arch or crossed bar, b) Footboard of bed, c) Foot/Lower leg, d) Ankle/foot orthotic positioning device. Here the Arched bar is added to a ankle foot orthotic to provide protection. The positions of the foot and the footboard relate that the patient can also be supine while wearing this device. The patient would be prevented from scratching or crossing the other leg on top of a fragile area or surgical site.

FIG. 2) Non-contacting roll bar shield attached to article of apparel: This figure represents the attachment of an arch or arches from the shoulder to the elbow area to prevent inadvertent or other mechanical trauma to an area of the upper are. This is a frequent site for a dialysis shunt but represents the possible protection of a site by a non surface contacting bar.

FIG. 2 Details: a) The area representing a surgical site or dialysis shunt, b) the arching bars of the roll bar are attached to clothing. By strapping, hook & loop, sliding into pockets or stitching, the bar can be affixed to clothing. In this application the grasping or otherwise contacting of the upper arm area would not contact a fragile surgical or shunt site and reduce the risk of failure.

What is claimed:

1. A mechanical trauma protection device which embodies and rigid skeletal frame by which to be attached over an open splint to protect an area of the human body from mechanical trauma comprising:
a multi-side rigid member extending from one end of the bodily injury to the opposing end having an elongated skeletal shape and protective resistance;
said mechanical trauma protection device having attaching points whereby the device is mechanically connected to either an existing cast or a portion of the human anatomy.

2. A mechanical trauma protection device as set forth in claim 1, wherein said protection device is constructed of resin based materials.

3. A mechanical trauma protection device as set forth in claim 1, wherein said protection device is constructed of metallic based materials.

4. A mechanical trauma protection device as set forth in claim 1, wherein said protection device is attached to a brace.

5. A mechanical trauma protection device as set forth in claim 1, wherein said protection device is incorporated into apparel.

6. A mechanical trauma protection device as set forth in claim 1, wherein said protection device is constructed of wood, leather or plant materials.
